(19) United States
(12) Patent Application Publication
(10) Pub. No.: US 2025/0037340 A1
(43) Pub. Date: Jan. 30, 2025

(54) AUTOMATICALLY GENERATING A VIDEO IN WHICH A PERSON SEES AND HEARS A REPRESENTATION OF THEMSELF DELIVERING A POSITIVE MESSAGE TAILORED TO NEGATIVE FEELINGS THEY ARE PRESENTLY EXPERIENCING

(71) Applicant: Providence St. Joseph Health, Renton, WA (US)

(72) Inventors: Srinath Malur Srinivasa, Hyderabad (IN); Sai Rahul Nagubandi, Hyderabad (IN); Cathlyn Fraguela Rios, Seattle, WA (US)

(21) Appl. No.: 18/360,698

(22) Filed: Jul. 27, 2023

(51) Int. Cl. G06T 13/20; G06F 40/56; G06T 13/40; G06V 40/30; G10L 13/02

(52) U.S. Cl. CPC G06T 13/205; G06F 40/56; G06T 13/40; G06V 40/30; G10L 13/02

(57) ABSTRACT

A facility for generating an audio-video sequence is described. The facility accesses one or more digital visual artifacts captured from the person's head, and uses them to create an animatable avatar. The facility receives input describing the person's emotional state, and generates a textual script conveying a positive message with respect to the person's described emotional state. The facility subjects the script to a text-to-speech tool to obtain a speech audio sequence reciting the script, and animates the avatar in a manner coordinated with the speech audio sequence to obtain a video sequence. The facility combines the speech audio sequence and the video sequence to obtain an audio-video sequence, and makes the audio-video sequence available to the person.

### BACKGROUND

When facing a behavioral health challenge—such as grief from the loss of a pet, depression from a breakup, or feelings of isolation following a move to a new city—it is common for a person to arrange to be treated by a mental health professional. These can include psychologists, psychiatrists, social workers, therapists, and counselors of other types, for example.

### BRIEF DESCRIPTION OF THE DRAWINGS

FIG. 1 is a block diagram showing some of the components typically incorporated in at least some of the computer systems and other devices on which the facility operates.

FIG. 2 is a flow diagram showing a process performed by the facility in some embodiments to compile script templates.

FIG. 3 is a table diagram showing sample contents of a script template table used by the facility in some embodiments to store script templates and their attributes.

FIG. 4 is a flow diagram showing a process performed by the facility in some embodiments to create a video for a person and present it to them.

FIG. 5 is a display diagram showing a first display presented by the facility in some embodiments to collect the information needed to create a video for a person.

FIG. 6 is a display diagram showing a second display presented by the facility in some embodiments in order to play a video generated for a person to the person.

### DETAILED DESCRIPTION

The inventors have recognized that treatment by a mental health professional can be burdensome to arrange; expensive; and time-consuming to participate in. These considerations tend to limit the set of people who seek treatment for behavioral health challenges. Additionally, treatment by a mental health professional can seem out-of-proportion for behavioral health challenges at the less serious end of the spectrum.

In response to recognizing these difficulties in and obstacles to obtaining treatment by mental health professional, the inventors have conceived and reduced to practice a software and/or hardware facility for automatically generating a video in which a person sees and hears a representation of themself delivering a positive message tailored to negative feelings they are presently experiencing (“the facility”).

In some embodiments, the facility uses one or more images of the person, together with details about the person such as age and sex/gender, to construct an animatable avatar for the person. The facility obtains from the person a description of how they are feeling, and automatically generates a positive textual script that is responsive to these feelings to be delivered to the person through the avatar. In particular, in some embodiments, the script portrays a version of the person whose thinking patterns and behaviors are stronger and more self-compassionate than the person's present state, and not struggling with present unhelpful self-talk and thought patterns.

In some embodiments, to generate this script, the facility selects from among a set of script templates prepared by mental health professionals or other human experts one that is appropriate to the described feelings, and uses the person's details and/or description to customize the script template. In some embodiments, the facility uses the description and the person's details to construct a prompt requesting that a generative language model such as GPT-4 or another large language model generate the script, and submits the constructed prompt to the generative language model. In some embodiments, the facility trains or refines this model using datasets prepared by mental health professionals to address range of behavioral issues or scenarios.

The facility then prepares a video in which it renders the script in both text-to-speech audio and synchronized video showing animation of the avatar to portray the speech with associated facial expressions, gestures, postural changes, etc. In some embodiments, the animation is biased toward physical expressions of mental balance and poise. The facility presents the prepared video to the person.

By operating in some or all of the ways described above, the facility gives the person a level of self-reassurance; based on providing the information to create their avatar once, the person can receive a new tailored message whenever they feel a need to mentally charge themselves up and get going with life, with renewed confidence in their own abilities.

Additionally, the facility improves the functioning of computer or other hardware, such as by reducing the dynamic display area, processing, storage, and/or data transmission resources needed to perform a certain task, thereby enabling the task to be permitted by less capable, capacious, and/or expensive hardware devices, and/or be performed with lesser latency, and/or preserving more of the conserved resources for use in performing other tasks. For example, by obviating treatment for some people, the facility conserves processor cycles and network traffic that would otherwise be devoted to operating a two-way video connection between these people and mental health professionals.

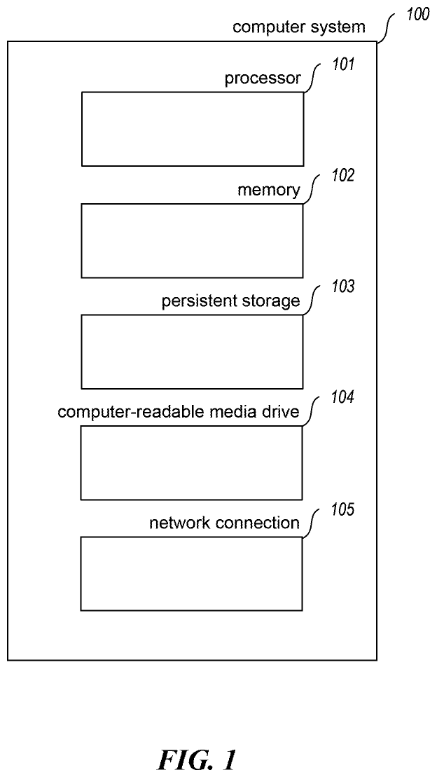

FIG. 1 is a block diagram showing some of the components typically incorporated in at least some of the computer systems and other devices on which the facility operates. In various embodiments, these computer systems and other devices 100 can include server computer systems, cloud computing platforms or virtual machines in other configurations, desktop computer systems, laptop computer systems, netbooks, mobile phones, personal digital assistants, televisions, cameras, automobile computers, electronic media players, etc. In various embodiments, the computer systems and devices include zero or more of each of the following: a processor 101 for executing computer programs and/or training or applying machine learning models, such as a CPU, GPU, TPU, NNP, FPGA, or ASIC; a computer memory 102 for storing programs and data while they are being used, including the facility and associated data, an operating system including a kernel, and device drivers; a persistent storage device 103, such as a hard drive or flash drive for persistently storing programs and data; a computer-readable media drive 104, such as a floppy, CD-ROM, or DVD drive, for reading programs and data stored on a computer-readable medium; and a network connection 105 for connecting the computer system to other computer systems to send and/or receive data, such as via the Internet or another network and its networking hardware, such as switches, routers, repeaters, electrical cables and optical fibers, light emitters and receivers, radio transmitters and receivers, and the like. While computer systems configured as described above are typically used to support the operation of the facility, those skilled in the art will appreciate that the facility may be implemented using devices of various types and configurations, and having various components.

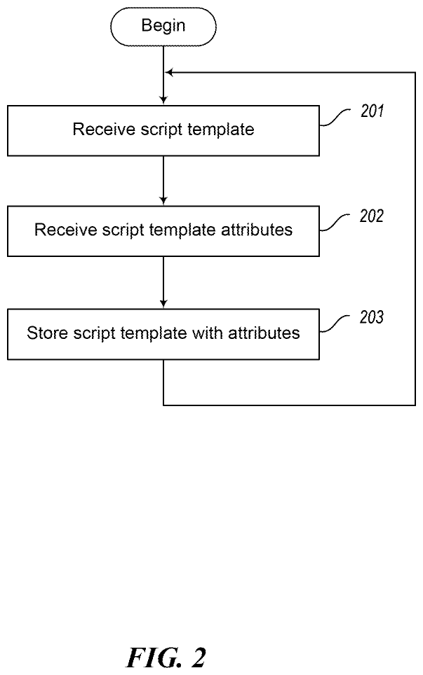

FIG. 2 is a flow diagram showing a process performed by the facility in some embodiments to compile script templates. In act 201, the facility receives a script template, such as a script template prepared by a mental health professional or other human expert. In some embodiments, the script template is designed for a particular mental health scenario, and includes fields and/or options that permit it to be tailored to a particular person based upon their details and their contemporaneous input about their present emotional state. In act 202, the facility receives one or more script attributes for the script template received in act 201. The script attributes characterize the situation or situations for which the script template is appropriate. In various embodiments, the script attributes are keywords, per-situation flags, nodes of a taxonomy, ontology, or other hierarchy of situations, etc. In act 203, the facility stores the script template received in act 201 together with its attributes received in step 202 to be available to use to generate particular scripts for particular people, such as in a script template table.

Those skilled in the art will appreciate that the acts shown in FIG. 2 and in each of the flow diagrams discussed below may be altered in a variety of ways. For example, the order of the acts may be rearranged; some acts may be performed in parallel; shown acts may be omitted, or other acts may be included; a shown act may be divided into subacts, or multiple shown acts may be combined into a single act, etc.

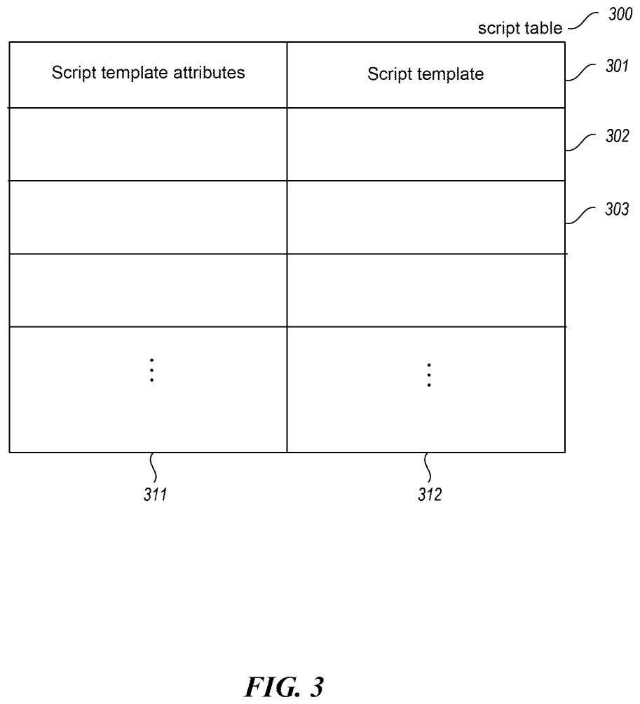

FIG. 3 is a table diagram showing sample contents of a script template table used by the facility in some embodiments to store script templates and their attributes. The script template table 300 is made up of rows, such as rows 301-303, which each correspond to a different script template available for use by the facility to generate a script for a particular person. Each row is divided into the following columns: a script template attributes column 311 containing attributes describing the script template to which the row corresponds that can be used to match that script template to a particular person in their situation; and a script template column 312 containing the textual content of the script template to which the row corresponds.

While FIG. 3 and each of the table diagrams discussed below show a table whose contents and organization are designed to make them more comprehensible by a human reader, those skilled in the art will appreciate that actual data structures used by the facility to store this information may differ from the table shown, in that they, for example, may be organized in a different manner; may contain more or less information than shown; may be compressed, encrypted, and/or indexed; may contain a much larger number of rows than shown, etc.

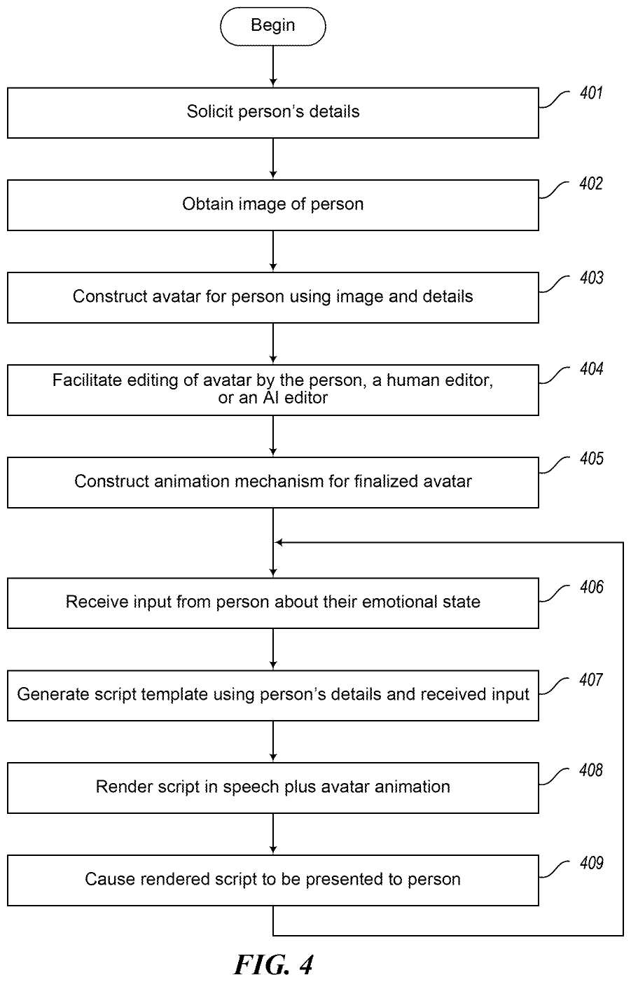

FIG. 4 is a flow diagram showing a process performed by the facility in some embodiments to create a video for a person and present it to them. In some embodiments, the facility recommends to the person that they engage in this process on the basis of automatically identifying a behavioral health challenge of the person, such as by observing written communications, spoken communications, voice tone, facial expressions, posture, time allocations and/or schedule nonadherence, etc.

In act 401, the facility solicits details about the person, such as details that can be used to identify situations that the person might find themselves in, and to tailor videos to them. For example, the person's sex may inform whether the person is experiencing a situation related to their own pregnancy. The person's sex and age may be useful in configuring a text-to-speech conversion that most closely mimics the person's voice. A level of education of the person may be used in configuring complexity of language used in the script.

In act 402, the facility obtains one or more images of the person, such as one or more still images, and/or one or more video sequences. In various embodiments, these images and/or video sequences are selected, uploaded, or captured in real time, such as by using a smartphone camera or web camera. In act 403, the facility constructs an avatar for the person that resembles them, using the one or more images obtained in act 402 and the person's details obtained in act 401. In act 404, the facility permits the avatar constructed in act 403 to be edited to be more accurate and/or lifelike, such as by the person themselves, another human editor, or an artificial intelligence editor. In act 405, the facility constructs an animation mechanism for the avatar finalized in act 404 that permits avatar to be moved and changed to reflect the facial movements of speech, and exhibit facial expressions, gestures, postural changes, etc.

In act 406, the facility receives input from the person about their present emotional state. This may include information describing their present feelings, as well as any perceived causes of those feelings or other antecedents that may be related. In various embodiments, the facility receives this input from the person via typing or speech. In some embodiments, the facility subjects the received input to an automatic language understanding tool—such as Microsoft LUIS—to discern information from the input including intents, entities, and sentiments present in the input. The facility uses this information as a basis for script generation.

In act 407, the facility generates a script using the person's details and received input. In some embodiments, the facility generates this script by selecting one of the script templates stored by the facility in the script template table, by matching script template attributes stored in the script template table for each script template with the person's details and input about emotional state. The facility then populates and/or customizes the selected script template for the person, such as by using the person's details and input to populates fields, choose among options, etc.

In some embodiments, the facility uses a generative language model such as GPT4 or another large language model to generate the script. In particular, the facility generates a prompt tailored to elicit a positive statement about self relating to a situation discerned from the person's received input. In various embodiments, the facility uses various kinds of generative language models, invoked in various ways. In various such embodiments, the facility uses a stock or standard generative language model; uses a generative language model trained from scratch on materials generated or selected by the designers or operators of the facility and/or mental health experts, in some cases including the script templates or example scripts; uses a stock or standard generative language model that is further trained with materials generated or selected by designers or operators of the facility, in some cases including script templates or example scripts; uses standard or stock generative language models that are fine-tuned using materials generated or selected by the designers and/or operators of the facility. In various embodiments, this fine tuning includes such approaches as updating the model's embedding layers; updating the model's language modeling head; updating parameters of the model; prompt engineering; prompt-tuning or optimization; reinforcement learning from human feedback, such as pre-training a language model, gathering data and training a reward model, or fine-tuning the language model with reinforcement learning. In some embodiments, the facility submits materials generated or selected by the designers and/or operators of the facility as part of the prompt that the facility submits to the generative language model.

In act 408, the facility renders the script generated in act 407 in speech—using text-to-speech and animation of the avatar performed in a way coordinated with the timing of the speech. In some embodiments, the facility performs the text-to-speech transformation using a generative speech model—such as the VALL-E neural codec language model generative speech model—that is capable of voice cloning, and submits the script to this generative speech model together with a sample of the person's voice, such that the voice audio sequence that is generated seems to have been spoken in the person's voice. In some embodiments, the facility subjects the script to a sentiment analysis tool—such as Lexalytics Semantria, Meaning Cloud Sentiment Analysis, Rosette Sentiment Analysis, or IBM Watson® Natural Language Understanding—to identify portions of the script in each of which a particular sentiment arises; the facility uses these identified sentiments to control aspects of the rendering of the video and/or audio to reflect these identified sentiments in voice tone and volume, gestures, facial expressions, postural changes, etc. In some embodiments, the facility uses an animation tool such as DeepMotion Animate 3D to animate facial expressions in coordination with the speech. In some embodiments, the facility uses an animation tool such as Adobe Mixamo to animate gestures and postural changes in coordination with the speech. In act 409, the facility causes the script rendered in act 408 to be presented to the person. After act 409, the facility continues in act 406 to receive input from the person about a later emotional state.

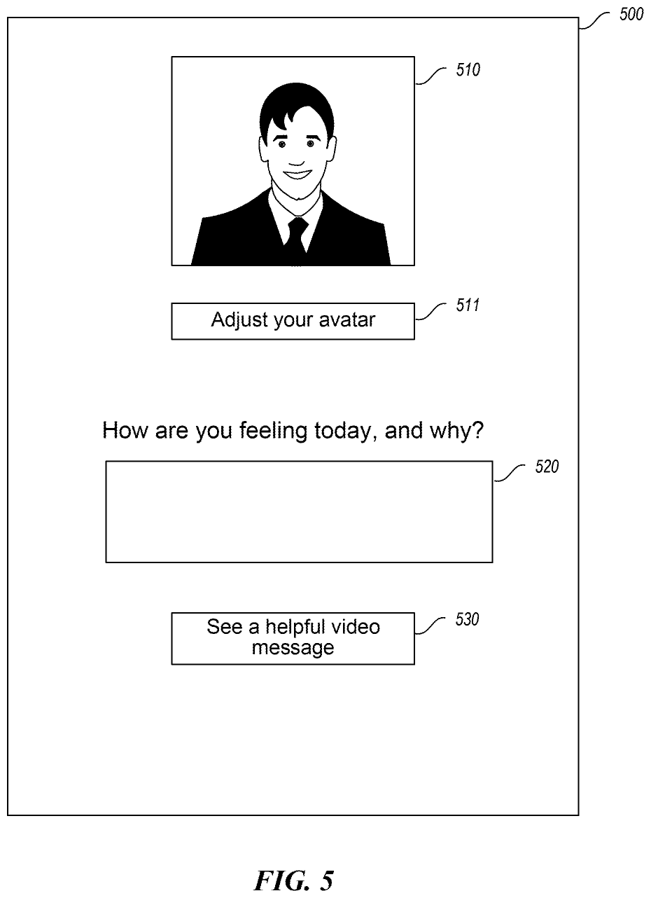

FIG. 5 is a display diagram showing a first display presented by the facility in some embodiments to collect the information needed to create a video for a person. In the display 500, a region 510 shows an avatar constructed by the facility for the person. The facility can activate control 511 in order to make adjustments and other edits to the avatar. The facility also contains a control 520 into which the user can enter input about their emotional state, as well as reasons for and/or antecedents of it. In various embodiments, the facility can enter this input by typing or speaking. The display also includes a control 530 that the person can activate in order to see a helpful video message delivered by the avatar that is responsive to their emotional state.

While FIG. 5 and each of the display diagrams discussed below show a display whose formatting, organization, informational density, etc., is best suited to certain types of display devices, those skilled in the art will appreciate that actual displays presented by the facility may differ from those shown, in that they may be optimized for particular other display devices, or have shown visual elements omitted, visual elements not shown included, visual elements reorganized, reformatted, revisualized, or shown at different levels of magnification, etc.

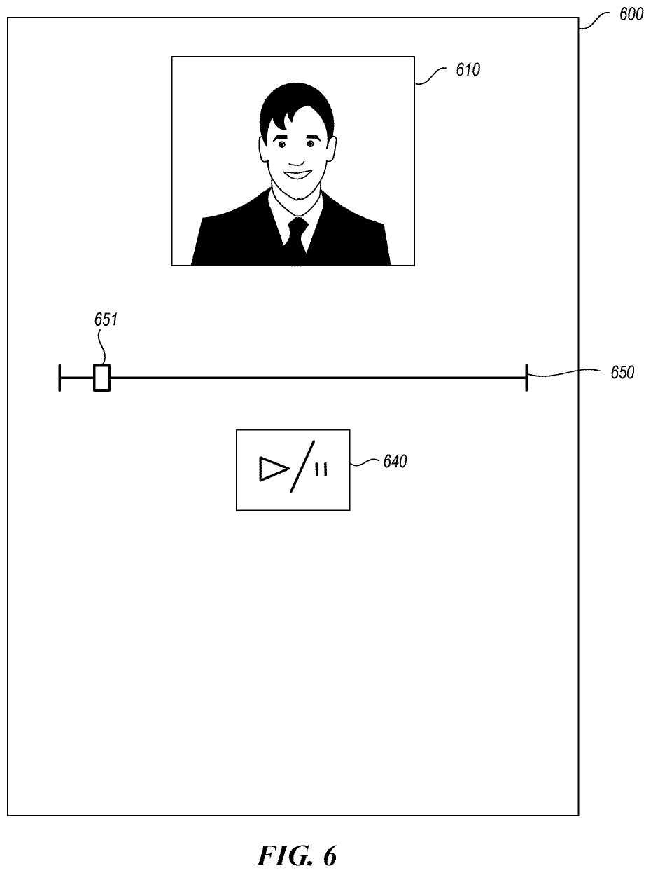

FIG. 6 is a display diagram showing a second display presented by the facility in some embodiments in order to play a video generated for a person to the person. The display 600 includes a video window 610 in which animation of the person's avatar is shown in coordination with playback of speech audio generated by subjecting the script generated by the facility to text-to-speech. The person can control playing verses pausing of the video using control 640, and view and change the position 651 in the duration of the video 650 by dragging slider 651.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method in a computing system, comprising:
receiving attributes of a person;
causing one or more digital visual artifacts to be captured from the person's head;
using the attributes and the one or more digital visual artifacts to create an animatable avatar;
receiving from the person input describing the person's emotional state;
generating a script conveying a positive message with respect to the person's described emotional state;
subjecting the script to a text-to-speech tool to obtain a speech audio sequence reciting the script;
animating the avatar in a manner coordinated with the speech audio sequence to obtain a video sequence;
combining the speech audio sequence and the video sequence to obtain an audio-video sequence; and
causing the audio-video sequence to be presented to the person.

2. The method of claim 1 wherein the digital visual artifacts are (a) one or more still images; (b) one or more video sequences, or (c) one or more still images and one or more video sequences.

3. The method of claim 1, further comprising:
invoking a natural language understanding tool, passing the input; and
receiving from the natural language understanding tool in response to the invocation at least one intent identified in the input,
wherein the identified intent is used in generating the script.

4. The method of claim 1 wherein the generating comprises:
accessing a script template selection resource identifying, for each of a plurality of script templates, attributes of the script template;
using the script template selection resource to select one of the plurality of script templates whose attributes best match (a) aspects of the attributes of the person, (b) aspects of the input, or (c) aspects of the attributes of the person and aspects of the input;
customizing the selected script template using (a) aspects of the attributes of the person, (b) aspects of the input, or (c) aspects of the attributes of the person and aspects of the input to obtain the script.

5. The method of claim 1 wherein the generating comprises:
constructing a prompt reflecting (a) aspects of the attributes of the person, (b) aspects of the input, or (c) aspects of the attributes of the person and aspects of the input;
invoking a generative language model, passing the prompt; and
receiving the script from the generative language model in response to the invocation.

6. The method of claim 1, further comprising:
receiving from the person audio constituting a sample of the person's speech, and wherein the text-to-speech tool to which the script is subjected is a generative speech model that is directed to clone the voice in the sample audio in rendering the script.

7. The method of claim 1, further comprising:
subjecting the script to a sentiment analysis tool to identify portions of the script in each of which an identified sentiment arises; and
controlling the avatar animation during the identified portions of the script using the identified sentiments with respect to:
facial expressions;
gestures;
postural changes;
facial expressions and gestures;
facial expressions and postural changes
gestures and postural changes; or
facial expressions, gestures, and postural changes.

8. One or more memories collectively storing a data structure, the data structure comprising:
state usable to transform attributes of a person and text describing an emotional state of the person into a textual script conveying a positive message with respect to the person's described emotional state.

9. The one or more memories of claim 8 wherein the state comprises a plurality of entries, each entry comprising:
a customizable script template; and
attributes of the script template usable to evaluate the level to which the script template matches particular text describing an emotional state of the person, such that the data structure is usable to select one of the script templates best matching particular text describing an emotional state of the person to customize to obtain the script.

10. The one or more memories of claim 8 wherein the state comprises:
a generative language model prompt template,
such that the generative language model prompt template is customizable using the attributes and text to use as a basis for invoking a generative language model to generate the script.

11. One or more instances of computer-readable media collectively having contents configured to cause a computing system to perform a method, the method comprising:
accessing one or more digital visual artifacts captured from the person's head;
using the one or more digital visual artifacts to create an animatable avatar;
receiving input describing the person's emotional state;
generating a script conveying a positive message with respect to the person's described emotional state;
subjecting the script to a text-to-speech tool to obtain a speech audio sequence reciting the script;
animating the avatar in a manner coordinated with the speech audio sequence to obtain a video sequence;
combining the speech audio sequence and the video sequence to obtain an audio-video sequence; and
making the audio-video sequence available to the person.

12. The one or more instances of computer-readable media of claim 11, further comprising:
invoking a natural language understanding tool, passing the input; and
receiving from the natural language understanding tool in response to the invocation at least one intent identified in the input,
wherein the identified intent is used in generating the script.

13. The one or more instances of computer-readable media of claim 11 wherein the generating comprises:
accessing a script template selection resource identifying, for each of a plurality of script templates, attributes of the script template;
using the script template selection resource to select one of the plurality of script templates whose attributes best match (a) aspects of the attributes of the person, (b) aspects of the input, or (c) aspects of the attributes of the person and aspects of the input;
customizing the selected script template using (a) aspects of the attributes of the person, (b) aspects of the input, or (c) aspects of the attributes of the person and aspects of the input to obtain the script.

14. The one or more instances of computer-readable media of claim 11 wherein the generating comprises:
constructing a prompt reflecting (a) aspects of the attributes of the person, (b) aspects of the input, or (c) aspects of the attributes of the person and aspects of the input;
invoking a generative language model, passing the prompt; and
receiving the script from the generative language model in response to the invocation.

15. The one or more instances of computer-readable media of claim 11, further comprising:
accessing a plurality of sample messages;
instantiating the generative language model; and
training the generative language model using the sample messages.

16. The one or more instances of computer-readable media of claim 11, further comprising:
accessing a plurality of sample messages;
accessing a pre-trained generative language model; and
further training the pre-trained generative language model using the sample messages to obtain the generative language model that is invoked.

17. The one or more instances of computer-readable media of claim 11, further comprising:
accessing a plurality of sample messages;
accessing a pre-trained generative language model; and
fine-tuning training the pre-trained generative language model using the sample messages to obtain the generative language model that is invoked.

18. The one or more instances of computer-readable media of claim 11, further comprising:
accessing a plurality of sample messages; and
referencing the sample messages in invoking the generative language model.

19. The one or more instances of computer-readable media of claim 11, further comprising:
receiving from the person audio constituting a sample of the person's speech, and wherein the text-to-speech tool to which the script is subjected is a generative speech model that is directed to clone the voice in the sample audio in rendering the script.

20. The one or more instances of computer-readable media of claim 11, further comprising:
subjecting the script to a sentiment analysis tool to identify portions of the script in each of which an identified sentiment arises; and
controlling the avatar animation during the identified portions of the script using the identified sentiments with respect to:
facial expressions;
gestures;
postural changes;
facial expressions and gestures;
facial expressions and postural changes
gestures and postural changes; or
facial expressions, gestures, and postural changes.
